(19) United States
(12) Patent Application Publication
(10) Pub. No.: US 2007/0049879 A1
(43) Pub. Date: Mar. 1, 2007

(54) Vesicovaginal incontinence device and method of use

(76) Inventor: J. Santiago Gutierrez, Laredo, TX (US)

Correspondence Address: John C. Cave;Gunn & Lee, P.C., Suite 1500, 700 N. St. Mary's St., San Antonio, TX, 78205, US

(21) Appl. No.: 11/217,538

(22) Filed: Sep. 1, 2005

(51) Int. Cl. A61F 5/44

(52) U.S. Cl. 604/330; 604/331

(57) ABSTRACT

A vesicovaginal incontinence device having a flexible rubber hemispherical funnel with an inlet port and an outlet port. A first end of a rubber drainage tube is contiguous with the outlet port. The second end of the rubber drainage tube is removably connected to a fluid reservoir. A clamping means is positioned on the drainage tube for regulating drainage within the drainage tube. The fluid reservoir has a securing means attached to it for securing the fluid reservoir to a user's leg. The fluid reservoir has an intake tube at one end that is removably connected to the second end of the drainage tube. This intake tube has a non-return valve contained within it to prevent backflow. The fluid reservoir also contains an outlet tube at its lowermost end for draining fluid from the reservoir. The outlet tube has a one way valve for releasing urine from the reservoir.

### BACKGROUND OF THE INVENTION

1. Field of the Invention

Applicant's invention relates to a vesicovaginal incontinence device. More specifically, the present invention relates to a vesicovaginal incontinence device for collection of urine from the vagina due to incontinence from a vesicovaginal fistula.

2. Background Information

In a healthy female, the urinary system removes urea from the blood. Urea is carried in the bloodstream to the kidneys. The kidneys remove urea from the blood through filtering units called nephrons. Each nephron consists of a ball of small blood capillaries called the glomerulus and a small tube called a renal tube. Urea, together with water and other waste substances, forms the urine as it passes through the nephrons and down the renal tubes of the kidney. From the kidneys, urine travels down two tubes called ureters to the bladder. Circular muscles in the bladder called sphincters keep the urine from leaking. When the sphincter muscles relax, urine exits the bladder through the urethra. Urinary incontinence, often referred to as loss of bladder control, is the involuntary passage of urine. This incontinence; however, is generally leakage through the urethra. This problem can be treated with both medications and surgery.

The present invention however does not concern incontinence through the urethra, but rather incontinence through the vagina resulting from a vesicovaginal fistula. A vesicovaginal fistula (VVF) is an abnormal opening in the vesicovaginal tissue extending between the urinary bladder and the vagina. This fistula forms after certain traumatic events, such as those caused by complications in surgery, radiation, or cancer, disturb the region. As a result of the opening, urine continuously drains from the urinary bladder into the vaginal cavity beyond the woman's control.

The hallmark symptom of all vesicovaginal fistulae is constant urinary drainage into the vaginal vault and a subsequent incontinence due to the vagina's inability to control bladder function. The amount of urine that presents itself is a function of the fistula's size. Women with smaller fistulas sometimes exhibit periods of intermittent wetness while those with large fistulae experience almost a constant stream of urine. In fact, if the fistula is large enough, the woman may not be able to void her bladder at all due to the diminished pressure gradient from the drainage. Other symptoms include recurrent cystitis (bladder infection), perineal skin irritation due to constant wetness, vaginal fungal infections, and occasionally pelvic pain.

According to the United Nations, some two million women currently live with a vesicovaginal fistula and as many as one hundred thousand new cases arise each year. Without repair, a vesicovaginal fistula can be physically debilitating creating a significant impact on a woman's mobility. In addition to the physical symptoms, the psychological effects of a vesicovaginal fistula can be disastrous. The severe incontinence destroys a woman's normal functioning in society. In less industrialized cultures, a woman afflicted with this condition faces abandonment by her husband, loses her status and dignity, and becomes a social outcast. As a result of familial and social rejection, coupled with humiliation, many women with fistula live for years without any financial or social support since they are unable to make a living by themselves and have been ostracized. Understandably, some women cannot handle the effects of this condition and eventually resort to suicide.

The main cause of vesicovaginal fistulae largely corresponds to the technological climate of the woman's resident country. In developing countries with poor obstetric care, the majority of vesicovaginal fistulae occur after prolonged obstructed labor. The constant impaction of the fetus in the pelvis causes widespread tissue edema, hypoxia, tissue death (necrosis), and sloughing off. In other words, the constant pressure on the soft tissues of the vagina, bladder base, and urethra prohibits these sensitive tissues from maintaining a normal blood supply and they die due to lack of oxygen. As the tissue sloughs off, an abnormal opening appears in the vesicovaginal region. Vesicovaginal fistulae due to prolonged obstructed labor are generally larger than those of other origins and present themselves within the first twenty-four to forty-eight hours after the woman passes the fetus.

In developed countries, where obstetrics have evolved, prolonged labor is not a problem. Women from developed countries have different mindsets and opportunities than those from developing countries. Developed societies encourage women to seek out healthcare during the course of their entire pregnancy and obstetricians attack potential birth problems proactively. Once a doctor determines that labor is too difficult for a mother, the doctor can perform a Cesarean section to prevent fistula formation.

Ironically medical advancements in developed countries have nearly eradicated obstetric vesicovaginal fistulae, while different medical breakthroughs have become their main cause. Unintended consequences from gynecological surgery have become the primary sources of vesicovaginal fistulae in developed countries. Full abdominal hysterectomy is the most common gynecological surgery instigating this condition. Although debated, some analysts estimate that as many as two percent of full abdominal hysterectomies lead to a vesicovaginal fistula. Commentators attribute up to eighty percent of the total fistulae in developed countries to the full abdominal hysterectomy. Other causes are reduced blood supply due to radiation-induced necrosis, tumors in the vesicovaginal area, and other ureteric injuries. Symptoms for fistulae triggered by gynecological surgery usually occur within the first thirty days after the surgery, whereas radiation-induced fistulae develop over a much longer interval—forming anywhere from thirty days to thirty years after the antecedent event.

Since there is currently no device available to drain urine from the vagina, patients must wear adult diapers or sanitary pads to absorb the leakage. Catheterization through the urinary bladder can be used as a treatment. In this procedure, doctors insert a catheter and maintain it for up to thirty days. The catheter position, known as a Foley placement, allows urine to drain out of the bladder through the catheter, as opposed to draining through the fistula. In limited cases, the diversion of urine from the fistula allows the vesicovaginal tissue to repair itself and eliminates future leakage. However, the success rate for this treatment is minimal. Generally, this type of treatment cures only small fistulae-those less than five millimeters in length—, diagnosed within a few days of the triggering event, and non-radiation-induced. Catheterization does not work to eliminate the leakage through the vagina when a large fistula is involved. Because most vesicovaginal fistulae do not meet these criteria, the majority of them require surgical treatment.

Within the surgical realm, doctors have many options, including: (1) the vaginal approach, (2) the abdominal approach, (3) electrocauterization, (4) fibrin glue, (5) electrocauterization with endoscopic closure using fibrin glue and bovine collagen, (6) the laparoscopic approach, and (7) using interposition tissue flaps or grafts. Although differing methods exist, they share a common element. Most doctors will not perform any type of corrective surgery until the inflammation caused by the triggering event subsides. Dissipation of the swelling helps ensure proper diagnosis and allows doctors to formulate an appropriate treatment plan. Recommended wait times vary from one year after the initial diagnosis for radiation-induced fistula, to six months for fistula that result from a gynecological surgery. During this period of time patients continue to exhibit the embarrassing symptoms of vaginal incontinence.

Having a vesicovaginal fistula is truly a crippling condition. The World Health Organization has described a fistula as the single most dramatic aftermath of neglected childbirth. Thankfully, time-tested surgical methods exist to enable the proper repair of the vesicovaginal tissue. As long as the woman can maintain her dignity while waiting for the inflammation of the triggering event to subside, the condition is treatable. The present device was designed to give self esteem and mobility back to patients with VVF by providing a way for urine to be drained from the vagina.

### SUMMARY OF THE INVENTION

The preferred embodiment of the present invention includes a flexible rubber hemispherical funnel, having an inlet port and an outlet port. A first end of a rubber drainage tube is contiguous with the outlet port of the funnel. The second end of the rubber drainage tube is removably connected to a fluid reservoir. A clamping means is positioned on the drainage tube for regulating drainage within the drainage tube.

The fluid reservoir has an adjustable securing means, preferably a belt, attached to it for securing the fluid reservoir to a user's leg. In addition, the fluid reservoir has an intake tube at one end that is removably connected to the second end of the drainage tube. This intake tube has a non-return valve contained within it to prevent backflow. The fluid reservoir also contains an outlet tube at its lowermost end for draining fluid from the reservoir. The outlet tube has a one way valve for releasing urine from the reservoir.

The method of using the preferred embodiment of the present invention to drain urine due to a vesicovaginal fistula includes inserting the flexible rubber hemispherical funnel vaginally past the pubic bone and below the vesicovaginal fistula. The urine from the fistula is collected into the inlet port of the funnel. The urine drains from the funnel into the first end of the drainage tube by way of an outlet port on the funnel. The urine is collected into a fluid reservoir. When the urine within the fluid reservoir reaches a certain volume, the fluid reservoir can be emptied through the one way valve. The clamping means positioned on the drainage tube regulates drainage of the urine within the drainage tube.

### BRIEF DESCRIPTION OF THE DRAWINGS

FIG. 1 is a perspective view of the preferred embodiment of the present invention.

FIG. 2 is a perspective view of the fluid reservoir for the preferred embodiment of the present invention.

FIG. 3 is a perspective view of the preferred embodiment of the present invention as shown placed within a female vagina.

FIG. 4 is a perspective view of the fluid reservoir of the preferred embodiment of the present invention as shown attached to a user's leg.

### DETAILED DESCRIPTION OF THE PREFERRED EMBODIMENT

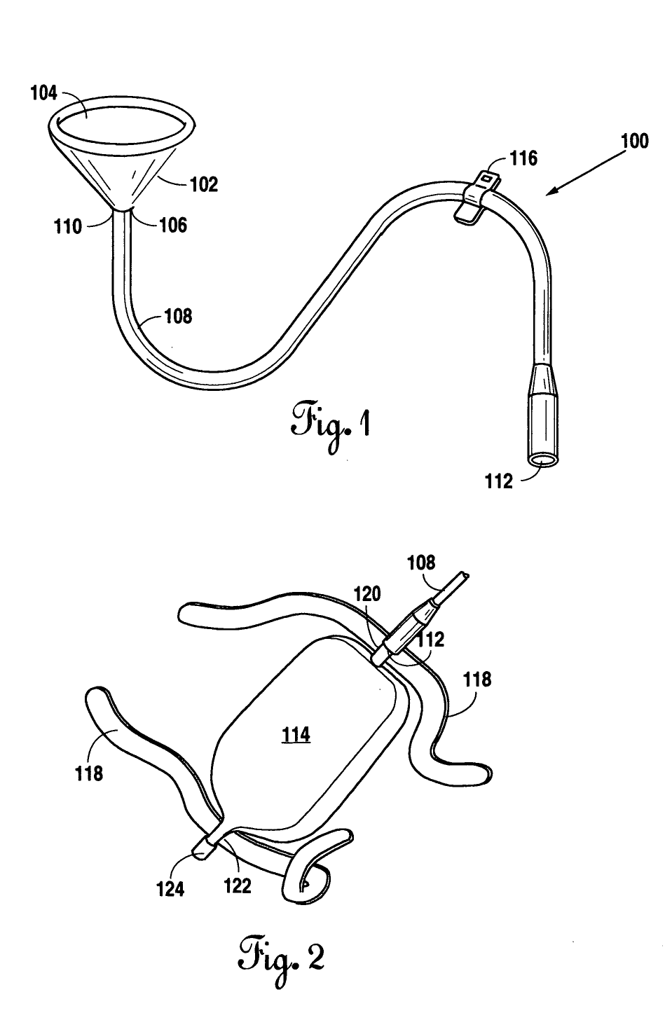

In FIG. 1, a perspective view of the preferred embodiment 100 of the present invention is shown. A flexible rubber hemispherical funnel 102 is shown having an inlet port 104 and an outlet port 106. A first end 110 of a drainage tube 108 is contiguous with the outlet port 106 of the funnel 102. The drainage tube 108 is preferably medical grade rubber. The second end 112 of the drainage tube 108 is removably connected to a fluid reservoir 114 (See FIG. 2). A clamping means 116 is positioned on the drainage tube 108. The clamping means 116 regulates drainage within the drainage tube 108.

FIG. 2 shows a perspective view of the fluid reservoir 114 for the preferred embodiment of the present invention. The fluid reservoir 114 has an adjustable securing means 118, preferably a belt, attached to it for securing the fluid reservoir 114 to the user's leg. The fluid reservoir 114 is preferably soft and light weight with a non-woven backing to allow the skin to breathe and feel comfortable. In addition, the fluid reservoir 114 has an intake tube 120 at one end that is removably connected to the second end 112 (See FIG. 1) of drainage tube 108 (See FIG. 1). This intake tube 120 has a non-return valve (not shown) contained within it to prevent backflow from the fluid reservoir 114 into the drainage tube 108 (See FIG. 1). It is also anticipated that the non-return valve (not shown) could be positioned in the drainage tube 108 near its second end 112. The fluid reservoir 114 also contains an outlet tube 122 at its lowermost end for draining fluid from the fluid reservoir 114. The outlet tube 122 has a one way valve (not shown) for releasing urine from the fluid reservoir 114 into an applicable vessel or waste container. A drainage cap 124 (See FIG. 4) is generally kept at the end of outlet tube 122 to stop leakage.

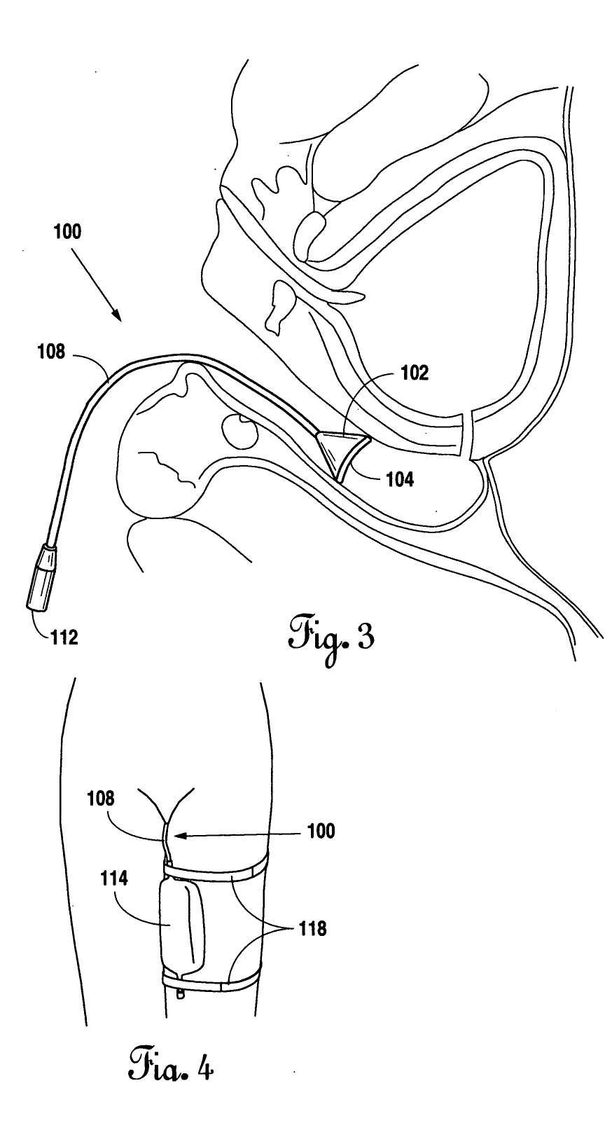

FIG. 3 is a perspective view of the preferred embodiment 100 of the present invention as shown placed within a female vagina. The flexible rubber hemispherical funnel 102 is inserted vaginally past the pubic bone and below the vesicovaginal fistula. Referring to FIG. 4, the fluid reservoir 114 is secured to the user's leg by way of the securing means 118. Care must be taken to ensure the fluid reservoir 114 is kept below the level of the bladder. The urine from the fistula is collected into the inlet port 104 of the funnel 102. The urine drains from the funnel 102 into the first end 110 (See FIG. 1) of drainage tube 108 by way of outlet port 106 (See FIG. 1) of the funnel 102.

Still referring to FIG. 4, the urine is collected into the fluid reservoir 114. When the urine within the fluid reservoir 114 reaches a certain volume, the fluid reservoir 114 can be emptied through the one way valve (not shown). The fluid reservoir 114 should be emptied at regular intervals, whenever it is half full and at bedtime. When draining the fluid reservoir 114, the user will unfasten the securing means 118 from her leg. Next, the user will unfasten the drainage tube 108 from the fluid reservoir 114. The clamping means 116 (See FIG. 1) positioned on the drainage tube 108 regulates drainage of the urine within the drainage tube 108. When the drainage tube 108 is removed from the fluid reservoir 114, the clamping means 116 (See FIG. 1) is situated on the drainage tube 108 and tightened in place to prevent leakage of urine out of the drainage tube 108. Once the clamping means 116 (See FIG. 1) is securely in place, the drainage cap 124 is removed. The urine from the fluid reservoir 114 can then be drained into an applicable vessel or waste container. Once the fluid reservoir 114 is emptied, the drainage cap 124 is replaced and the clamping means 116 (See FIG. 1) is released from the drainage tube 108. The fluid reservoir 114 is then reconnected to the drainage tube 108 and the securing means 118 is refastened and readjusted to the user's leg.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A vesicovaginal incontinence device comprising:
a funnel, said funnel having an inlet port and an outlet port;
a drainage tube, said drainage tube having a first end and a second end, said first end contiguous with said outlet port of said funnel;
a clamping means positioned on said drainage tube for regulating drainage within said drainage tube; and
a fluid reservoir removably connected to said second end of said drainage tube.

2. The vesicovaginal incontinence device of claim 1 further comprising a securing means attached to said fluid reservoir for securing said fluid reservoir to a user.

3. The vesicovaginal incontinence device of claim 1 wherein said fluid reservoir comprises an intake tube removably connected to said second end of said drainage tube.

4. The vesicovaginal incontinence device of claim 3 wherein said fluid reservoir comprises an outlet tube at its lowermost end for draining fluid from said fluid reservoir.

5. The vesicovaginal incontinence device of claim 3 wherein said intake tube comprises a non-return valve therein.

6. The vesicovaginal incontinence device of claim 3 wherein said drainage tube comprises a non-return valve therein.

7. The vesicovaginal incontinence device of claim 5 wherein said funnel is flexible.

8. The vesicovaginal incontinence device of claim 6 wherein said funnel is hemispherical.

9. A method of using a vesicovaginal incontinence device to drain urine from a woman's vaginal cavity comprising the steps of:
inserting a funnel of said device vaginally past the pubic bone of a user below the vesicovaginal fistula;
collecting urine into an inlet port of said funnel;
draining said urine from said funnel into a first end of a drainage tube by way of an outlet port on said funnel;
collecting said urine into a fluid reservoir; and
emptying said fluid reservoir.

10. The method of using a vesicovaginal incontinence device to drain urine due to a vesicovaginal fistula of claim 8 further comprising the step of regulating drainage of urine within said drainage tube.

11. The method of using a vesicovaginal incontinence device to drain urine due to a vesicovaginal fistula of claim 9 further comprising the step of securing said fluid reservoir to a user.
